(19) United States
(12) Patent Application Publication
(10) Pub. No.: US 2020/0188478 A1
(43) Pub. Date: Jun. 18, 2020

(54) PRE-MIXED, READY TO USE VANCOMYCIN COMPOSITIONS

(71) Applicant: FTF PHARMA PRIVATE LIMITED, Ahmedabad (IN)

(72) Inventors: Dhaval CHANDARANA, Dhrangadhra (IN); Sandip MEHTA, Ahmedabad (IN); Manish UMRETHIA, Ahmedabad (IN); Rutul PATEL, District-Mehsana (IN); Hardik PRAJAPATI, Ahmedabad (IN); Malay PATEL, Ahmedabad (IN); Jayanta Kumar MANDAL, Ahmedabad (IN)

(73) Assignee: FTF PHARMA PRIVATE LIMITED, Ahmedabad (IN)

(21) Appl. No.: 16/712,079

(22) Filed: Dec. 12, 2019

(30) Foreign Application Priority Data
Dec. 13, 2018 (IN) 201821047149

(51) Int. Cl. A61K 38/14; A61K 47/18; A61K 47/32; A61K 47/02; A61K 47/10; A61K 47/26; A61K 9/00

(52) U.S. Cl. CPC A61K 38/14; A61K 47/183; A61K 47/32; A61K 9/0019; A61K 47/10; A61K 47/26; A61K 47/02

(57) ABSTRACT

Pre-mixed, ready-to-use injectable compositions possess certain advantages such as convenience and ease of use as compared to an ampule formulation, improved safety for patients due to elimination of dosage errors and solution contamination, reduction of medical waste, and ease of administration in emergency situations. Pre-mixed, ready-to-use Vancomycin injectable preparations though marketed have numbers of disadvantages which makes its handling and use difficult. The present invention therefore provides pre-mixed, ready-to-use injectable formulations of Vancomycin which eliminates disadvantages and difficulties of the marketed product and at the same time maintains desired stability for prolonged time.

### FIELD OF THE INVENTION

The invention as disclosed in the instant specification is related to the field of pharmaceuticals, especially injectable preparations for intravenous use. Certain drugs are known to have administration as infusion. Therefore before administration, if the drug formulation is in the form of a concentrate liquid, it requires dilution using appropriate diluent or needs to get reconstituted first and then diluted using appropriate diluent in case of powder for reconstitution. Both processes may involve (i) stability concerns of the diluted solutions and (ii) dosing errors & cross contamination while diluting with diluent before administration to patient. It is therefore advantageous to have pre-mixed, ready-to-use injectable solution which does not require reconstitution or dilution before administration. Vancomycin is such a drug which is known to be administered through infusion and available (i) as powder for reconstitution and (ii) as frozen preparation which needs to be thawed before administration. The present invention is therefore related to such pre-mixed, ready-to-use Vancomycin injectable preparation which neither requires reconstitution and dilution nor requires to be thawed before administration.

### BACKGROUND OF THE INVENTION

Vancomycin hydrochloride is a tricyclic glycopeptide antibiotic drug derived from Amycolatopsis orientalis (formerly Nocardia orientalis). The molecular formula of Vancomycin hydrochloride is C₆₆H₇₅Cl₂N₉O₂₄.HCl and the molecular weight is 1485.71 gm/mol. Vancomycin hydrochloride has the following structural formula:

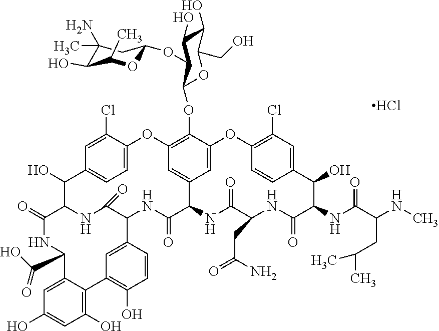

Vancomycin is indicated for the treatment of serious or severe infections caused by susceptible strains of methicillin-resistant (beta-lactam-resistant) staphylococci. It is indicated for penicillin-allergic patients, for patients who cannot receive or who have failed to respond to other drugs, including the penicillins or cephalosporins, and for infections caused by Vancomycin-susceptible organisms that are resistant to other antimicrobial drugs. Vancomycin is indicated for initial therapy when methicillin resistant staphylococci are suspected, but after susceptibility data are available, therapy should be adjusted accordingly.

Vancomycin is effective in the treatment of staphylococcal endocarditis. Its effectiveness has been documented in other infections due to staphylococci, including septicemia, bone infections, lower respiratory tract infections, skin and skin structure infections. When staphylococcal infections are localized and purulent, antibiotics are used as adjuncts to appropriate surgical measures.

Vancomycin has been reported to be effective alone or in combination with an aminoglycoside for endocarditis caused by Streptococcus viridans or S. bovis. For endocarditis caused by enterococci (e.g., E. faecalis), vancomycin has been reported to be effective only in combination with an aminoglycoside.

Vancomycin has been reported to be effective for the treatment of diphtheroid endocarditis. Vancomycin has been used successfully in combination with either rifampin, an aminoglycoside, or both in early-onset prosthetic valve endocarditis caused by S. epidermidis or diphtheroids.

Specimens for bacteriologic cultures should be obtained in order to isolate and identify causative organisms and to determine their susceptibilities to Vancomycin.

To reduce the development of drug-resistant bacteria and maintain the effectiveness of Vancomycin and other antibacterial drugs, Vancomycin should be used only to treat or prevent infections that are proven or strongly suspected to be caused by susceptible bacteria. When culture and susceptibility information are available, they should be considered in selecting or modifying antibacterial therapy. In the absence of such data, local epidemiology and susceptibility patterns may contribute to the empiric selection of therapy.

Vancomycin is contraindicated in patients with known hypersensitivity to this antibiotic. Solutions containing dextrose may be contraindicated in patients with known allergy to corn or corn products.

Vancomycin available as Vancomycin hydrochloride is approved in various dosage forms, such as capsule, powder for oral solution, injectable, powder for intravenous or IV (infusion). Vancomycin injections are available as concentrate liquid (for further dilution before intravenous administration), powder (for reconstitution and further dilution before intravenous administration) and ready-to-use, pre-mixed injection as frozen preparation.

Pre-mixed, ready-to-use injectable formulation of Vancomycin hydrochloride packaged in plastic container was first approved in 1993 by USFDA (Vancocin hydrochloride in plastic container by Baxter Healthcare Corporation). Thereafter, two more strengths of pre-mixed, ready-to-use injectable formulations of Vancomycin hydrochloride were approved by USFDA in 1999 and 2010 respectively. The Vancocin hydrochloride in plastic container is frozen, iso-osmotic, sterile, non-pyrogenic 100 mL, 150 mL, or 200 mL solution containing 500 mg, 750 mg, or 1 g Vancomycin respectively as Vancomycin hydrochloride packaged in the GALAXY plastic container. Each 100 mL of solution contains approximately 5 g of dextrose hydrous, USP or 0.9 g of sodium chloride, USP. The pH of the solution may have been adjusted with hydrochloric acid and/or sodium hydroxide. Thawed solutions have a pH in the range of 3.0 to 5.0. After thawing to room temperature, this solution is intended for intravenous use only.

This GALAXY container is fabricated from a specially designed multilayer plastic (PL 2040). Solutions are in contact with the polyethylene layer of this container and can leach out certain chemical components of the plastic in very small amounts within the expiration period. The suitability of the plastic has been confirmed in tests in animals according to USP biological tests for plastic containers as well as by tissue culture toxicity studies.

However, the product leaflet of pre-mixed, ready-to-use Vancomycin injection gives following instructions regarding how to use frozen injections:
(a) The Vancomycin injection should be stored at or below −20° C.
(b) The frozen containers should be thawed at room temperature (at 25° C.) or under refrigeration (at 5° C.).
(c) The frozen product should not be thawed by immersion in water baths or by microwave irradiation.
(d) The frozen product must not be forcibly thawed.
(e) The thawed product must not be refreeze.
(f) The thawed solution remains chemically stable for 72 hours (3 days) at room temperature or for 30 days when stored under refrigeration.
(g) The containers may be fragile in the frozen state therefore handling of containers must be done with care.

From above it can be seen that though the ready-to-use, pre-mixed Vancomycin injections are available in the market, use of such injection formulations is bound by some limitations. Therefore the need still exists in the art for making such formulations available which overcome above mentioned limitations. The inventors of the present invention through their continuous efforts for catering need of the art, have now surprisingly found that pre-mixed, ready-to-use Vancomycin injections are stable in liquid state and that too without freezing the solution. Following are few advantages of the presently invented Vancomycin injection formulations:
(a) The Vancomycin formulation of the present invention is not required to store at −20° C.
(b) The Vancomycin formulation of the present invention is not in frozen state therefore it does not require thawing before administration to the patient.
(c) The risk associated with thawing procedure (as mentioned in the points (c) and (d) above) has been eliminated by means of the Vancomycin formulation of the present invention.
(d) The Vancomycin formulation of the present invention is stable for prolonged time, say for example at least for three months or more. Further, no special storage conditions (such as storing at −20° C.) are required to achieve the prolonged stability.
(e) The Vancomycin formulation of the present invention is not in the frozen state, therefore containers containing the vancomycin formulation of the present invention may not be fragile. Therefore no special care for handling the containers is required.

US 20180133286 filed by Xellia Pharmaceuticals ApS, incorporated herein by reference in its entirety, discloses an aqueous pharmaceutical composition comprising Vancomycin, N-acetyl-D-Alanine, an amino acid, wherein amino acid is selected from Glycine, Alanine, Serine, Leucine, Valine, Lysine, Arginine and Ornithine, preferably L-Lysine and D-Lysine. US'286 publication also discloses an aqueous pharmaceutical composition comprising Vancomycin, N-acetyl-D-Alanine and an organic solvent selected from ethanol and polyethylene glycol. US'286 publication also discloses an aqueous pharmaceutical composition comprising Vancomycin, N-acetyl-D-Alanine, wherein the pharmaceutical composition has a pH of about 3-6; wherein the pharmaceutical composition comprises an amino acid selected from D-Serine, D-Leucine, D-Valine, L-Lysine, D-Lysine, L-Arginine, D-Ornithine and L-Ornithine; and wherein the pharmaceutical composition further comprises a pharmaceutically acceptable organic solvent. However, in the pre-mixed solutions disclosed by US'286 publication, N-acetyl-D-alanine is an essential ingredient used for stabilization of the Vancomycin in aqueous solutions.

US 20170348385 filed by Xellia Pharmaceuticals ApS, incorporated herein by reference in its entirety, discloses a pharmaceutical composition comprising a glycopeptide antibiotic (i.e. Vancomycin); an excipient selected from N-acetyl-D-Alanine and N-acetyl-Glycine; and an amino acid, wherein the amino acid is selected from Glycine, Alanine, Serine, Leucine, Valine, Lysine, Arginine and Ornithine, preferably L-Lysine and D-Lysine. Upon reading specification of US '385 publication it seems that N-acetyl-D-alanine or N-acetyl-glycine is an essential excipient which stabilizes Vancomycin in the aqueous solution.

WO 2017194385 filed by Xellia Pharmaceuticals ApS, incorporated herein by reference in its entirety, discloses a liquid pharmaceutical composition comprising a glycopeptide antibiotic (i.e. Vancomycin) and sulfobutylether-betacyclodextrin.

US 20170304396 filed by Latitude Pharmaceuticals Inc., incorporated herein by reference in its entirety, discloses an injectable solution composition comprising Vancomycin at a concentration of about 0.1% w/v to about 12% w/v and tryptophan having an empirical formula of C₁₁H₁₂N₂O₂ selected from the group consisting of the L-form, the D-form, a mixture of the L- and D-forms or salts thereof, at a concentration between about 0.1% w/v to 2.5% w/v, wherein tryptophan inhibits formation of crystalline degradation product-1 (CDP-1). US '396 publication teaches that amino acids such as methionine, alanine, cysteine, arginine, proline and asparagine have no or negative effect on vancomycin stability in solution.

US 20150314002 filed by Insmed Inc., incorporated herein by reference in its entirety, discloses a stabilized lipid-based glycopeptide antibiotic composition comprising: a lipid component; a glycopeptide antibiotic component (i.e. Vancomycin); and an amino acid or derivative thereof, wherein the amino acid or the derivative thereof reduces the degradation rate of the glycopeptide antibiotic, and wherein the antibiotic composition is at least 44% more stable than an antibiotic composition comprising the same lipid component and the same glycopeptide antibiotic component, that does not comprise an amino acid or a derivative thereof.

US 20170202906 filed by SciDose LLC, incorporated herein by reference in its entirety, discloses a vancomycin-containing composition, comprising about 25 mg/mL to about 150 mg/mL vancomycin or a pharmaceutically acceptable salt thereof; and a pharmaceutically acceptable fluid having a pH of about 3 to about 8, comprising a polar solvent comprising propylene glycol, polyethylene glycol or mixtures thereof; and lactic acid, lactate or mixtures thereof per mL of pharmaceutically acceptable fluid.

US 20160101147 filed by SciDose LLC, incorporated herein by reference in its entirety, discloses a vancomycin-containing composition, comprising about 25 mg/mL to about 150 mg/mL vancomycin or a pharmaceutically acceptable salt thereof; and a pharmaceutically acceptable fluid having a pH of about 3 to about 8, comprising a polar solvent comprising propylene glycol, polyethylene glycol or mixtures thereof; and lactic acid, lactate or mixtures thereof per mL of pharmaceutically acceptable fluid.

US 20170224767 filed by SciDose LLC, incorporated herein by reference in its entirety, discloses a liquid vancomycin-containing composition, consisting of from about 50 mg/mL to about 250 mg/mL of vancomycin or a pharmaceutically acceptable salt thereof, glycerol, a member of the group consisting of lactic acid, lactate or mixtures thereof, a base and a pharmaceutically acceptable fluid containing water, wherein the liquid vancomycin composition has a pH of from about 4.5 to about 7.5.

Literature survey of Vancomycin pharmaceutical compositions reveals that the preparation of different types of formulations of Vancomycin e.g. oral liquid formulations, dry powder/freeze-dried formulations, topical formulations, liposomal formulations, has been tried by formulation scientists over a period of time. However, ready-to-use, pre-mixed Vancomycin solution for injection is not explored much. Further, the ready-to-use, pre-mixed Vancomycin injectable solutions known in the prior art involves use of specific excipients to stabilize Vancomycin in aqueous solutions such as N-acetyl-D-alanine, N-acetyl-glycine, tryptophan, di & tri-peptides of amino acids etc. None of the prior art teaches effect of methionine as well as other stabilizing agents disclosed herein this specification alone or in combination with methionine on stability of vancomycin in solutions. Therefore, the need still exists in the art to prepare ready-to-use, pre-mixed aqueous solutions of Vancomycin for injection which despite involves use of known and commonly available excipients, provides desired stability to the Vancomycin formulations.

### SUMMARY OF THE INVENTION

Dilution of the drug product available as either concentrate liquid or powder for reconstitution for infusion administration involves certain issues, e.g. stability concerns of the diluted product, concerns regarding storage conditions of the diluted products, dosing errors & cross contamination because of dilution, compatibility of various diluents with the drug products, etc. In one embodiment, the present invention therefore provides pre-mixed, ready-to-use, injectable preparations of Vancomycin which eliminates abovementioned concerns from consideration.

As mentioned in the foregoing paragraphs, the pre-mixed, ready-to-use Vancomycin injectable preparations available in the market possess certain limitations. In another embodiment, the present invention therefore provides such pre-mixed, ready-to-use Vancomycin injectable formulations which eliminate limitations belong to the marketed ready-to-use, pre-mixed Vancomycin injectable preparations.

Diluted drug products may not be stable for prolonged time. Therefore the diluted product, if not used, may not be stored beyond its storage period and may need to discard and discontinue from use. Further, the marketed ready-to-use, pre-mixed Vancomycin formulation is a frozen preparation and requires thawing before administration. Such thawed solution may also not be stored for prolonged time and cannot be refreeze. Furthermore, the frozen preparation is required to be stored at −20° C. In another embodiment, the present invention therefore provides those Vancomycin injectable preparations which are stable at 5° C.±3° C. for prolonged time, e.g. at least for 3 months or more.

In another embodiment, the present invention provides pre-mixed, ready-to-use Vancomycin injectable formulations which despite involve use of commonly known and available excipients, provide desired stability to the Vancomycin formulations. The compositions of the present invention does not include stabilizing agents such as N-acetyl-D-alanine, N-acetyl-glycine and Tryptophan.

In another embodiment, the present invention provides method of preparation of the pre-mixed, ready-to-use Vancomycin injectable preparations.

In another embodiment, the present invention provides pre-mixed, ready-to-use Vancomycin injectable formulations for the treatment of septicaemia, infective endocarditis, skin and skin structure infections, bone infections, lower respiratory tract infections in adults and pediatric patients (neonates and older).

In another embodiment, the present invention provides use of pre-mixed, ready-to-use Vancomycin injectable formulation for its intended purposes.

Described herein are pre-mixed, ready-to-use Vancomycin injectable preparations comprising Vancomycin or its pharmaceutically acceptable salt and at least one pharmaceutically acceptable excipient. The pharmaceutically acceptable excipient for use in the preparation of the Vancomycin injectable preparations according to the present invention is selected from the group consisting of a tonicity modifying agent, an anti-oxidant, a buffering agent, a pH adjusting/modifying agent, a stabilizing agent, a chelating agent, a solvent/co-solvent/solubilizer, a surfactant and a vehicle.

In another embodiment, the present invention provides pre-mixed, ready-to-use Vancomycin injectable preparations comprising Vancomycin or its pharmaceutically acceptable salt, one or more tonicity modifying agents, one or more stabilizing agents, one or more buffering agents, one or more pH adjusting agents and a vehicle.

In another embodiment, the present invention provides pre-mixed, ready-to-use Vancomycin injectable preparations comprising Vancomycin or its pharmaceutically acceptable salt, a tonicity modifying agent, an anti-oxidant, a buffering agent, a pH adjusting agent and a vehicle.

In another embodiment, the present invention provides pre-mixed, ready-to-use Vancomycin injectable preparations comprising Vancomycin or its pharmaceutically acceptable salt, a tonicity modifying agent, an anti-oxidant, a pH adjusting agent and a vehicle.

In another embodiment, the present invention provides pre-mixed, ready-to-use Vancomycin injectable preparations comprising Vancomycin or its pharmaceutically acceptable salt, a tonicity modifying agent, an anti-oxidant, a buffering agent and a vehicle.

In another embodiment, the present invention provides pre-mixed, ready-to-use Vancomycin injectable preparations comprising Vancomycin or its pharmaceutically acceptable salt, a tonicity modifying agent, a buffering agent, a pH adjusting agent and a vehicle.

In another embodiment, the present invention provides pre-mixed, ready-to-use Vancomycin injectable preparations comprising Vancomycin or its pharmaceutically acceptable salt, a tonicity modifying agent, a pH adjusting agent and a vehicle.

In another embodiment, the present invention provides pre-mixed, ready-to-use Vancomycin injectable preparations comprising Vancomycin or its pharmaceutically acceptable salt, a tonicity modifying agent, a buffering agent, and a vehicle.

In another embodiment, the present invention provides pre-mixed, ready-to-use Vancomycin injectable preparations comprising Vancomycin or its pharmaceutically acceptable salt, one or more stabilizers, one or more buffers and/or pH adjusting agents and a vehicle.

In another embodiment, the present invention provides pre-mixed, ready-to-use Vancomycin injectable preparations comprising Vancomycin or its pharmaceutically acceptable salt, one or more solubilizers, one or more buffers and/or pH adjusting agents and a vehicle.

In another embodiment, the present invention provides pre-mixed, ready-to-use Vancomycin injectable preparations comprising Vancomycin or its pharmaceutically acceptable salt, one or more stabilizers, one or more solubilizers, one or more tonicity modifying agents, one or more buffers and/or pH adjusting agents and a vehicle.

In another embodiment, the present invention provides pre-mixed, ready-to-use Vancomycin injectable preparations comprising Vancomycin or its pharmaceutically acceptable salt, one or more chelating agents, one or more buffers and/or pH adjusting agents and a vehicle.

In another embodiment, the present invention provides pre-mixed, ready-to-use Vancomycin injectable preparations comprising:
(i) vancomycin or its pharmaceutically acceptable salt;
(ii) a stabilizing agent selected from methionine, polyvinylpyrrolidone and a mixture thereof; and
(iii) a vehicle selected from water such as water for injection.

In another embodiment, the present invention provides pre-mixed, ready-to-use Vancomycin injectable preparations comprising:
(i) vancomycin or its pharmaceutically acceptable salt;
(ii) a solubilizer selected from polyethylene glycol, propylene glycol, poloxamer and a mixture thereof; and
(iii) a vehicle selected from water such as water for injection.

In another embodiment, the present invention provides pre-mixed, ready-to-use Vancomycin injectable preparations comprising:
(i) vancomycin or its pharmaceutically acceptable salt;
(ii) a stabilizing agent selected from methionine, polyvinylpyrrolidone and a mixture thereof;
(iii) a solubilizer selected from polyethylene glycol, propylene glycol, poloxamer and a mixture thereof;
(iv) a tonicity modifying agent selected from dextrose, mannitol, sodium chloride and a mixture thereof; and
(v) a vehicle selected from water such as water for injection.

In another embodiment, the present invention provides pre-mixed, ready-to-use Vancomycin injectable preparations comprising:
(i) vancomycin or its pharmaceutically acceptable salt;
(ii) a chelating agent selected from edetate disodium (disodium EDTA), edetate disodium (disodium EDTA) anhydrous, edetate sodium, edetate calcium disodium, edetate calcium di sodium anhydrous, edetic acid and a mixture thereof;
(iii) a vehicle selected from water such as water for injection; and
(iv) optionally a pharmaceutically acceptable excipient.

In another embodiment, the present invention provides pre-mixed, ready-to-use Vancomycin injectable preparations comprising:
(i) vancomycin or its pharmaceutically acceptable salt;
(ii) a stabilizing agent selected from methionine, polyvinylpyrrolidone and a mixture thereof;
(iii) a tonicity modifying agent selected from dextrose, mannitol, sodium chloride and a mixture thereof; and
(iv) a vehicle selected from water such as water for injection.

The pre-mixed, ready-to-use Vancomycin injectable preparations described herein may also comprise a buffering agent and/or a pH adjusting agent. The pre-mixed, ready-to-use Vancomycin injectable preparations described herein may also comprise a pharmaceutically acceptable excipient.

In some embodiments, the pH of the Vancomycin injectable preparation of the present invention is between about 3.0 and about 7.0. In some of the embodiments, the pH of the Vancomycin injectable preparation of the present invention is between about 4.0 and about 6.0. In some of the further embodiments, pH of the Vancomycin injectable preparation of the present invention is between about 4.0 and about 5.0. In some of the further embodiments, pH of the Vancomycin injectable preparation of the present invention is between about 4.5 and about 5.5. In some of the further embodiments, pH of the Vancomycin injectable preparation of the present invention is between about 5.0 and about 6.0. The pH between about 3.0 and about 7.0 is meant to include any pH value from 3.0 to 7.0 including pH 3.0 and including pH 7.0 and any pH value in between 3.0 and 7.0. Similarly, the pH between about 4.0 and about 6.0 is meant to include any pH value from 4.0 to 6.0 including pH 4.0 and including pH 6.0 and any pH value in between 4.0 and 6.0. The pH between about 4.0 and about 5.0 is meant to include any pH value from 4.0 to 5.0 including pH 4.0 and including pH 5.0 and any pH value in between 4.0 and 5.0. The pH between about 4.5 and about 5.5 is meant to include any pH value from 4.5 to 5.5 including pH 4.5 and including pH 5.5 and any pH value in between 4.5 and 5.5. The pH between about 5.0 and about 6.0 is meant to include any pH value from 5.0 to 6.0 including pH 5.0 and including pH 6.0 and any pH value in between 5.0 and 6.0.

In some embodiments, the present invention also provides process for the preparation of pre-mixed, ready-to-use Vancomycin injectable preparations.

In some embodiments, the present invention also provides use of the pre-mixed, ready-to-use Vancomycin injectable formulations for the treatment of septicaemia, infective endocarditis, skin and skin structure infections, bone infections, lower respiratory tract infections in adults and pediatric patients (neonates and older).

### DETAILED DESCRIPTION OF THE INVENTION

The concentrated vial formulations or powder for reconstitution of Vancomycin hydrochloride may present numbers of issues for patients and health care professionals, e.g.
(a) the requirement of dilution can result in a lag time that prevents a patient in an acute setting from receiving the drug in a timely fashion. After an order is written by a physician in a hospital setting, it can sometimes take up to 2 hours or longer for the reconstituted product to be available to the patient. This results in the health care professional facing the choice of either a dangerous delay in administration of a potentially life-saving therapeutic agent, or replacing the preferred formulation with a less effective therapy that would be available in a more timely fashion;
(b) the breaking of the vial neck may result in exposing the patient to glass contamination of the product and exposing health care professional to an increased risk of injuring themselves when handling and breaking the glass vials;
(c) there is an increased probability of dosing errors by requiring health care professionals to dilute the product. Such errors may manifest themselves as an overdose or an underdose if the product is not diluted properly. Likewise there is an additional possibility that the concentrated form will be administered “as is” which is contraindicated and can result in adverse events;
(d) the diluted form must not be stored beyond storage period due to stability issues;
(e) the selection of an inappropriate diluent can have an adverse effect on the stability and could cause breakdown of the drug.

Therefore it is advantageous to have pre-mixed, ready-to-use injectable pharmaceutical compositions available for administration to the patient. Additional benefits of the pre-mixed, ready-to-use, injectable pharmaceutical compositions include convenience and ease of use as compared to a vial formulation, improved safety for patients due to elimination of dosage errors and solution contamination, reduction of medical waste, and ease of administration in emergency situations.

The pre-mixed, ready-to-use Vancomycin injectable formulations have been made available in the market but these formulations are associated with numbers of disadvantages such as,
(a) The marketed Vancomycin injection should be stored at or below −20° C.
(b) The frozen containers should be thawed at room temperature (at 25° C.) or under refrigeration (at 5° C.).
(c) The frozen product should not be thawed by immersion in water baths or by microwave irradiation.
(d) The frozen product must not be forcibly thawed.
(e) The thawed product must not be refreeze.
(f) The thawed solution remains chemically stable for 72 hours (3 days) at room temperature or for 30 days when stored under refrigeration.
(g) The containers may be fragile in the frozen state therefore handling of containers must be done with care.

Further, the ready-to-use, pre-mixed Vancomycin injectable solutions known in the prior art involves use of specific excipients to stabilize Vancomycin in aqueous solutions. Therefore, the need still exists in the art to prepare ready-to-use, pre-mixed aqueous solutions of Vancomycin for injection which despite involves use of commonly available excipients, provides desired stability to the Vancomycin formulations. The need also exists for providing those Vancomycin injectable formulations which are pre-mixed, ready-to-use and at the same time overcome abovementioned disadvantages. In one of the principal embodiments, the present invention therefore provides pre-mixed, ready-to-use Vancomycin injectable formulations which overcome disadvantages associated with the marketed as well as prior known pre-mixed Vancomycin formulations.

The pre-mixed, ready-to-use Vancomycin injectable preparations according to the present invention comprise Vancomycin or its pharmaceutically acceptable salt, one or more tonicity modifying agents, one or more stabilizing agents, one or more buffering agents, one or more pH adjusting agents and a vehicle.

In some of the alternative embodiments, the pre-mixed, ready-to-use Vancomycin injectable preparations according to the present invention comprise Vancomycin or its pharmaceutically acceptable salt, a tonicity modifying agent, an anti-oxidant, a buffering agent, a pH adjusting agent and a vehicle.

In some of the alternative embodiments, the pre-mixed, ready-to-use Vancomycin injectable preparations according to the present invention comprise Vancomycin or its pharmaceutically acceptable salt, a tonicity modifying agent, an anti-oxidant, a pH adjusting agent and a vehicle.

In some of the alternative embodiments, the pre-mixed, ready-to-use Vancomycin injectable preparations according to the present invention comprise Vancomycin or its pharmaceutically acceptable salt, a tonicity modifying agent, an anti-oxidant, a buffering agent and a vehicle.

In some of the alternative embodiments, the pre-mixed, ready-to-use Vancomycin injectable preparations according to the present invention comprise Vancomycin or its pharmaceutically acceptable salt, a tonicity modifying agent, a buffering agent, a pH adjusting agent and a vehicle.

In some of the alternative embodiments, the pre-mixed, ready-to-use Vancomycin injectable preparations according to the present invention comprise Vancomycin or its pharmaceutically acceptable salt, a tonicity modifying agent, a pH adjusting agent and a vehicle.

In some of the alternative embodiments, the pre-mixed, ready-to-use Vancomycin injectable preparations according to the present invention comprise Vancomycin or its pharmaceutically acceptable salt, a tonicity modifying agent, a buffering agent, and a vehicle.

In some of the alternative embodiments, the present invention provides pre-mixed, ready-to-use Vancomycin injectable preparations comprising Vancomycin or its pharmaceutically acceptable salt, one or more stabilizers, one or more buffers and/or pH adjusting agents and a vehicle.

In some of the alternative embodiments, the present invention provides pre-mixed, ready-to-use Vancomycin injectable preparations comprising Vancomycin or its pharmaceutically acceptable salt, one or more solubilizers, one or more buffers and/or pH adjusting agents and a vehicle.

In some of the alternative embodiments, the present invention provides pre-mixed, ready-to-use Vancomycin injectable preparations comprising Vancomycin or its pharmaceutically acceptable salt, one or more stabilizers, one or more solubilizers, one or more tonicity modifying agents, one or more buffers and/or pH adjusting agents and a vehicle.

In some of the alternative embodiments, the present invention provides pre-mixed, ready-to-use Vancomycin injectable preparations comprising Vancomycin or its pharmaceutically acceptable salt, one or more chelating agents, one or more buffers and/or pH adjusting agents and a vehicle.

In some of the alternative embodiments, the present invention provides pre-mixed, ready-to-use Vancomycin injectable preparations comprising:
(i) vancomycin or its pharmaceutically acceptable salt;
(ii) a stabilizing agent selected from methionine, polyvinylpyrrolidone and a mixture thereof;
(iii) a vehicle selected from water; and
(iv) optionally a pharmaceutically acceptable excipient selected from a buffering agent and/or a pH adjusting agent.

In some of the alternative embodiments, the present invention provides pre-mixed, ready-to-use Vancomycin injectable preparations comprising:
(i) vancomycin or its pharmaceutically acceptable salt;
(ii) a solubilizer selected from polyethylene glycol, propylene glycol, poloxamer and a mixture thereof;
(iii) a vehicle selected from water; and
(iv) optionally a pharmaceutically acceptable excipient selected from a buffering agent and/or a pH adjusting agent.

In some of the alternative embodiments, the present invention provides pre-mixed, ready-to-use Vancomycin injectable preparations comprising:
(i) vancomycin or its pharmaceutically acceptable salt;
(ii) a stabilizing agent selected from methionine, polyvinylpyrrolidone and a mixture thereof;
(iii) a solubilizer selected from polyethylene glycol, propylene glycol, poloxamer and a mixture thereof;
(iv) a tonicity modifying agent selected from dextrose, mannitol, sodium chloride and a mixture thereof;
(v) a vehicle selected from water; and
(vi) optionally a pharmaceutically acceptable excipient selected from a buffering agent and/or a pH adjusting agent.

In some of the alternative embodiments, the present invention provides pre-mixed, ready-to-use Vancomycin injectable preparations comprising:
(i) vancomycin or its pharmaceutically acceptable salt;
(ii) a chelating agent selected from edetate disodium (disodium EDTA), edetate disodium (disodium EDTA) anhydrous, edetate sodium, edetate calcium disodium, edetate calcium disodium anhydrous, edetic acid and a mixture thereof; and
(iii) a vehicle selected from water; and
(iv) optionally a pharmaceutically acceptable excipient.

In some of the alternative embodiments, the present invention provides pre-mixed, ready-to-use Vancomycin injectable preparations comprising:
(i) vancomycin or its pharmaceutically acceptable salt;
(ii) a stabilizing agent selected from methionine, polyvinylpyrrolidone and a mixture thereof;
(iii) a tonicity modifying agent selected from dextrose, mannitol, sodium chloride and a mixture thereof;
(iv) a vehicle selected from water; and
(v) optionally a pharmaceutically acceptable excipient selected from a buffering agent and/or a pH adjusting agent.

In some of the alternative embodiments, the present invention provides pre-mixed, ready-to-use Vancomycin injectable preparations comprising:
(i) vancomycin or its pharmaceutically acceptable salt; and
(ii) at least one of the following:
   (a) a stabilizer selected from methionine, polyvinylpyrrolidone and a mixture thereof;
   (b) a solubilizer/co-solvent selected from polyethylene glycol, poloxamer and a mixture thereof;
   (c) a tonicity modifying agent selected from mannitol, dextrose, sodium chloride and a mixture thereof;
   (d) a chelating agent selected from edetate disodium (disodium EDTA), edetate disodium (disodium EDTA) anhydrous, edetate sodium, edetate calcium disodium, edetate calcium disodium anhydrous, edetic acid and a mixture thereof;
   (e) a buffering agent and/or a pH adjusting agent; and
   (f) any combination of (a), (b), (c), (d) and (e).

In some of the alternative embodiments, the present invention also provides pre-mixed, ready-to-use Vancomycin compositions which comprise vancomycin or its pharmaceutically acceptable salt, one or more tonicity modifying agents and one or more buffering agents and/or one or more pH adjusting agents. These compositions do not include methionine or any other excipient as stabilizing agent.

In some of the alternative embodiments, the present invention also provides Vancomycin injection compositions which are concentrate liquids. These compositions comprise Vancomycin or its pharmaceutically acceptable salt and one or more pharmaceutically acceptable excipients. The liquid concentrate compositions of the present invention are ready to dilute compositions. Such compositions may be diluted before administering to the patient with an appropriate diluent such as, for example, WFI (water for injection), 0.9% sodium chloride, or 5% dextrose or other diluent which is pharmaceutically and clinically acceptable.

In some of the alternative embodiments, the present invention provides Vancomycin injectable preparations which are concentrated liquids comprising:
(i) vancomycin or its pharmaceutically acceptable salt; and
(ii) at least one of the following:
   (a) a stabilizer selected from methionine, polyvinylpyrrolidone and a mixture thereof;
   (b) a solubilizer/co-solvent selected from polyethylene glycol, poloxamer and a mixture thereof;
   (c) a tonicity modifying agent selected from mannitol, dextrose, sodium chloride and a mixture thereof;
   (d) a chelating agent selected from edetate disodium (disodium EDTA), edetate disodium (disodium EDTA) anhydrous, edetate sodium, edetate calcium disodium, edetate calcium disodium anhydrous, edetic acid and a mixture thereof;
   (e) a buffering agent and/or a pH adjusting agent; and
   (f) any combination of (a), (b), (c), (d) and (e).

In some of the alternative embodiments, the present invention also provides Vancomycin compositions which are liquid concentrates comprising vancomycin or its pharmaceutically acceptable salt, one or more tonicity modifying agents and one or more buffering agents and/or one or more pH adjusting agents. These compositions do not include methionine or any other excipient as stabilizing agent.

In one of the further embodiments, the present invention provides stable Vancomycin formulations. The compositions of the present invention are stable for prolonged time, e.g. for at least 3 months or more, for at least 6 months or more, or for at least one year or more, when stored under various storage conditions.

In one of the further embodiments, the formulations of the present invention are stable for prolonged time when stored under storage conditions. The term “storage conditions” as used herein without limitation include typical storage conditions such as 2° C.-8° C. The compositions of the present invention may also be stored at 40° C.±2° C./75±5% RH, 30° C.±2° C./65±5% RH, 25° C.±2° C./40±5% RH, 25° C.±2° C./60±5% RH, 40° C.±2° C./NMT 25% RH (NMT=not more than) and accelerated conditions such as 40° C.±2° C./75±5% RH. The term “prolonged time” as used herein indicates that the premixed formulations of the present invention are stable for at least 1 month or more, at least 3 months or more, at least 6 months or more or at least 12 months or more when stored under storage conditions.

In one of the further embodiments, the present invention provides method of using ready-to-use, premixed injectable formulations of Vancomycin or pharmaceutically acceptable salt thereof for treating one or more disorders which can be treated by administration of Vancomycin or pharmaceutically acceptable salt thereof.

The premixed formulations of the present invention comprise 0.05-15 mg/mL Vancomycin or a pharmaceutically acceptable salt thereof. For example, suitable concentrations of Vancomycin or a pharmaceutically acceptable salt thereof, include, but are not limited to 0.05-0.1 mg/mL, 0.1-15 mg/mL, 0.1-10 mg/mL, 0.1-5 mg/mL, 0.1-3.0 mg/mL, 0.1-2.0 mg/mL, or 0.1-1.0 mg/mL. In some of the alternative embodiments, the formulations of the present invention comprise about 0.1 mg/mL to about 50 mg/mL Vancomycin or a pharmaceutically acceptable salt thereof. The range includes any value from 0.1 mg/mL to 50 mg/mL including 0.1 mg/mL and including pH 50 mg/mL and any value in between 0.1 mg/mL and 50 mg/mL.

In some of the embodiments, the premixed formulations of the present invention comprise Vancomycin hydrochloride as the active ingredient at a concentration sufficient to permit intravenous administration at a concentration between 0.05 mg/mL and 15 mg/mL. In some embodiments, the concentration of Vancomycin hydrochloride suitable for use in the compositions and methods of the present invention includes, but is not limited to, at least about 5 mg/mL.

In some of the further embodiments, the premixed formulations of the present invention comprise, in addition to Vancomycin or its pharmaceutically acceptable salts, a buffering agent and/or a pH adjusting agent that has sufficient buffering/pH adjusting capacity to maintain the desired pH range throughout the storage period of the product. Accordingly, suitable pH ranges for use in the premixed formulations of the present invention is between about 3.0 and about 6.0. The range includes any pH value from 3.0 to 6.0 including pH 3.0 and including pH 6.0 and any pH value in between 3.0 and 6.0. In some embodiments, the pH of the premixed formulations is between about 4.5 and about 5.5. The range includes any pH value from 4.5 to 5.5 including pH 4.5 and including pH 5.5 and any pH value in between 4.5 and 5.5. In some embodiments, the pH of the premixed formulations is between about 4.0 and about 5.0. The range includes any pH value from 4.0 to 5.0 including pH 4.0 and including pH 5.0 and any pH value in between 4.0 and 5.0. In some embodiments, the pH of the premixed formulations is between about 5.0 and about 6.0. The range includes any pH value from 5.0 to 6.0 including pH 5.0 and including pH 6.0 and any pH value in between 5.0 and 6.0.

Buffers suitable for use in the premixed formulations of the present invention include, but are not limited to, pharmaceutically acceptable salts and acids of acetate, glutamate, citrate, tartrate, benzoate, lactate, histidine or other amino acids, gluconate, phosphate, malate, succinate, formate, propionate, and carbonate. “Pharmaceutically acceptable” is used herein in the sense of being compatible with the other ingredients of the formulation and not deleterious to the recipient thereof. Accordingly, the term “pharmaceutically acceptable salt” references salt forms of the active compounds which are prepared with counter ions which are non-toxic under the conditions of use and are compatible with a stable formulation. Buffers suitable for use in the premixed formulations of the present invention also include, but are not limited to tris (hydroxymethyl)aminomethane (TRIS), triethanolamine, etc.

The concentration of the buffer in the formulation can be expressed in mg/mL, g/L or as a molar concentration. Typically, from about 0.0001 mg/mL to about 100 mg/mL of a suitable buffer is present in the formulations of the present invention. Thus, the premixed formulations can comprise from about 0.0001 to about 0.001 mg/mL of a suitable buffer, from about 0.001 to about 0.01 mg/mL of a suitable buffer, from about 0.01 to about 0.1 mg/mL of a suitable buffer, from about 0.1 to 1 mg/mL of a suitable buffer, from about 1 to about 5 mg/mL of a suitable buffer, from about 5 to about 10 mg/mL of a suitable buffer, from about 10 to about 15 mg/mL of a suitable buffer, from about 15 to about 20 mg/mL of a suitable buffer, from about 20 to about 25 mg/mL of a suitable buffer, from about 25 to about 50 mg/mL of a suitable buffer, from about 50 to about 75 mg/mL of a suitable buffer, and from about 75 to about 100 mg/mL of a suitable buffer.

Alternatively, the buffer concentration can be expressed as molar concentrations. In typical embodiments, from about 0.1 to 100 mM of a suitable buffer is present in the pharmaceutical compositions. Thus, the premixed pharmaceutical compositions can comprise a suitable buffer having a concentration from about 0.1 to about 100 mM, from about 0.1 to about 0.5 mM, from about 0.5 to about 1.0 mM, from about 1.0 to about 5 mM, from about 5 to about 10 mM, from about 10 to about 15 mM, from about 15 to about 25 mM, from about 25 to about 50 mM, from about 50 to about 75 mM, and from about 75 to about 100 mM.

In some of the embodiments, the premixed formulations of the present invention further comprise a pH adjusting agent. Suitable pH adjusting agents typically include at least an acid or a salt thereof, and/or a base or a salt thereof. Acids and bases can be added on an as needed basis in order to achieve the desired pH. For example, if the pH is greater than the desired pH, an acid can be used to lower the pH to the desired pH. Acids suitable for use in premixed formulations include, but are not limited to, hydrochloric acid, phosphoric acid, citric acid, ascorbic acid, acetic acid, sulphuric acid, carbonic acid and nitric acid. By way of another example, if the pH is less than the desired pH, a base can be used to adjust the pH to the desired pH. Bases suitable for use in premixed formulations include, but are not limited to, sodium hydroxide, potassium hydroxide, calcium hydroxide, sodium carbonate, sodium citrate, sodium acetate, and magnesium hydroxide.

A “buffering agent” or a “pH adjusting agent” as used herein is a system which is used for the purposes and is capable of maintaining the desired/required pH of the formulations throughout desired/required time period, e.g. stability studies and/or shelf life of the drug product. The desired pH of the formulations according to the present invention is between about 3.0 and about 7.0, preferably between about 4.0 and about 6.0, more preferably between about 4.5 and about 5.5. The range includes any pH value from 4.5 to 5.5 including pH 4.5 and including pH 5.5 and any pH value in between 4.5 and 5.5.

In some of the embodiments of the invention, both buffering agent and pH adjusting agent are used. In some of the embodiments of the invention, only a buffering agent is used. In some of the embodiments of the invention, only a pH adjusting agent is used.

In some of the further embodiments, the premixed formulations further comprise one or more tonicity agents. Tonicity agents are used to adjust the osmolality of the premixed formulations to bring it closer to the osmotic pressure of body fluids, such as blood or plasma. In some embodiments, the tonicity of the premixed formulations can be modified by adjusting the concentration of buffer and/or other components present in the premixed formulations. Provided that the formulations of the present invention are physiologically compatible, the formulations do not require any particular osmolality. Therefore the formulations of the present invention can be hypotonic, isotonic or hypertonic. Typically, the premixed formulations of the present invention have a tonicity between about 250 and about 450 mOsm/kg.

In some of the alternative embodiments, the premixed formulations of the present invention are isotonic, i.e., in the range of 270-328 mOsm/kg. However, the formulations may have a tonicity in the range of 250-450 mOsm/kg. Therefore, the formulations may be either slightly hypotonic, 250-269 mOsm/kg, or slightly hypertonic, 329-450 mOsm/kg.

Suitable tonicity agents for use in the premixed formulations include, but are not limited to, anhydrous or hydrous forms of sodium chloride, dextrose, sucrose, xylitol, fructose, glycerol, sorbitol, mannitol, potassium chloride, mannose, calcium chloride, magnesium chloride and other inorganic salts. The quantity of the tonicity agent(s) in the formulation can be expressed in mg/mL or in g/L. Typically, the tonicity agent(s) is present from about 1 mg/mL to about 90 mg/mL. Thus, the premixed formulations can comprise one or more tonicity agents at about 1-5 mg/mL, at about 5-10 mg/mL, at about 10-15 mg/mL, at about 15-25 mg/mL, at about 25-50 mg/mL, at about 50-60 mg/mL, at about 60-70 mg/mL, at about 70-80 mg/mL, and at about 80 to 90 mg/mL, as well as combinations of the above ranges.

In alternative embodiments, the tonicity agent concentration is measured in weight/volume percent. In typical embodiments, the tonicity agent(s) is present from about 0.1% to about 10%. For example, suitable tonicity agent concentrations include, but are not limited to, from about 0.1% to about 0.2%, from about 0.2% to about 0.3%, from about 0.3% to about 0.4%, from about 0.4% to about 0.5%, from about 0.5% to about 0.6%, from about 0.6% to about 0.7%, from about 0.7% to about 0.8%, from about 0.8% to about 0.9%, from about 0.9% to about 1%, from about 1% to about 2%, from about 2% to about 3%, from about 3% to about 4%, from about 4% to about 5%, from about 5% to about 6%, from about 6% to about 7%, from about 7% to about 8%, from about 8% to about 9%, and from about 9% to about 10%, as well as combinations of the above ranges.

In some of the embodiments of the present invention, the tonicity agent is sodium chloride. Typically, the concentration of sodium chloride suitable for use in the premixed formulations of the present invention is between about 0.1% (w/v) to about 1.8%. By way of example, suitable sodium chloride concentrations include, but are not limited to, from about 0.1% to about 0.2%, from about 0.2% to about 0.3%, from about 0.3% to about 0.4%, from about 0.4% to about 0.5%, from about 0.5% to about 0.6%, from about 0.6% to about 0.7%, from about 0.7% to about 0.8% (which is equivalent to 8 mg/mL), from out 0.8% to about 0.9% (which is equivalent to 9 mg/mL), from about 0.9% to about 1.0%, from about 1% to about 1.2%, from 1.2% (which is equivalent to 12 mg/mL) to about 1.4%, from about 1.4% to about 1.6%, and from about 1.6% to about 1.8%.

In some of the embodiments of the present invention, the tonicity agent is dextrose. Typically, the concentration of dextrose suitable for use in the premixed formulations of the present invention is between about 2.5% (w/v) to about 7.5%. By way of example, suitable dextrose concentrations include, but are not limited to, from about 2.5% to about 3%, from about 3% to about 3.5%, from about 3.5% to about 4% (which is equivalent to about 40 mg/mL), from about 4% to about 4.5%, from about 4.5% to about 5% (which is equivalent to about 50 mg/mL), from about 5% to about 5.5%, from about 5.5% to about 6% (which is equivalent to about 60 mg/mL), from about 6% to about 6.5%, from about 6.5% to about 7%, as well as combinations of the above ranges.

In some of the further embodiments, the premixed formulations comprise two, three, four, or more tonicity agents. In some of the further embodiments, the premixed formulations comprise two tonicity agents. In some of the further embodiments, the premixed formulations of the present invention comprise combination of sodium chloride and dextrose. In such embodiments, the concentration of each tonicity agent is typically less than the concentration that is used when only a single agent is present in the premixed formulations.

In some of the further embodiments of the present invention, the premixed formulations may further comprise one or more co-solvents. A “co-solvent” is a solvent which is added to the aqueous formulation in a weight amount which is less than that of water and assists in the solubilization of Vancomycin and/or a pharmaceutically acceptable salt thereof, enhances stability of the premixed formulation, and/or adjusts the osmolality of the premixed pharmaceutical compositions. Co-solvents suitable for use in the premixed formulations include, but are not limited to, glycols (e.g., polyethylene glycol, propylene glycol), ethanol, poloxamer and polyhydric alcohols (e.g., sorbitol, mannitol, xylitol).

The quantity of the co-solvent used in the formulation can be expressed in mg/mL or in g/L. Typically, the co-solvent(s) is present from about 1 mg/mL to about 100 mg/mL. Thus, the premixed formulations can comprise one or more co-solvent(s) at about 1 to about 2 mg/mL, at about 2 to about 3 mg/mL, at about 3 to about 4 mg/mL, at about 4 to about 5 mg/mL, at about 5 to about 10 mg/mL, at about 10 to about 15 mg/mL, at about 15 to about 25 mg/mL, at about 25 to about 50 mg/mL, at about 50 to about 60 mg/mL, at about 60 to about 70 mg/mL, at about 70 to about 80 mg/mL, at about 80 to 90 mg/mL, and at about 90 to 100 mg/mL, as well as combination of the above ranges.

Alternatively, the co-solvent concentration is measured in weight/volume percent. Typically, the co-solvent(s) is present from about 0.1% to about 25%. For example, suitable co-solvent concentrations include, but are not limited to, at least about 0.1% to 0.3%, from about 0.3% to about 0.5%, from about 0.5% to about 0.7%, from about 0.7% to about 0.9%, from about 0.9% to about 1%, from about 1% to about 3%, from about 3% to about 5%, from about 5% to about 7%, from about 7% to about 9%, from about 9% to about 11%, from about 11% to about 13% from about 13% to about 15%, from about 15% to about 20%, and from about 20% to about 25%, as well as combination of the above ranges.

In some of the embodiments, “co-solvent” and “solubilizer/solubilizing agent” is used interchangeably and represents the same meaning and function.

Antioxidants can be compounds that can reduce a drug that has been oxidized, or compounds that are more readily oxidized than the agents they are to protect (oxygen scavengers). Many of the lipid-soluble antioxidants act as scavengers. Antioxidants can also act as chain terminators, reacting with free radicals in solution to stop the free-radical propagation cycle. Mixtures of chelating agents and antioxidants are often used because there appears to be a synergistic effect. This occurs because many of the agents act at differing steps in the oxidative process.

Some substances prone to oxidation include unsaturated oils/fats, compounds with aldehyde or phenolic groups, colors, flavors, sweeteners, plastics and rubbers, the latter being used in containers for products. Oxidation may manifest as products with an unpleasant odour, taste, appearance, precipitation, discoloration or even a slight loss of activity. The term rancidity refers to many typical off-flavors that result from autoxidation of unsaturated fatty acids that are present in oils and fats, and it affects many oils and fats. The distinct rancid odour may result from short-chain, volatile monomers resulting from the cleavage of the longer chain, less volatile oils and fats. Non-limiting examples of anti-oxidants are amino acids, α-Tocopherol acetate, Ascorbic acid, Erythorbic acid, Butylated hydroxytoluene (BHT), d-α-Tocopherol natural, Monothioglycerol, Sodium bisulfite, Sodium sulfite, Sodium metabisulfite, Potassium metabisulfite, Acetone sodium bisulfite, Ascorbyl palmitate, L-methionine, Cysteine, d-α-tocopherol synthetic, Nordihydroguaiaretic acid, Sodium formaldehyde sulfoxylate, Sodium thiosulfate, Acetylcysteine, Ascorbyl palmitate, Butylated hydroxyanisole (BHA), Cysteine hydrochloride, Dithiothreitol, Propyl gallate, Thiourea and the like or any combinations thereof.

Vehicles may be used in the liquid compositions of the present invention. Vehicles are the liquid bases that carry drugs and other excipients in dissolved or dispersed state. Vehicles may be aqueous or non-aqueous or mixture thereof. Non-aqueous solvents may also be added in the liquid compositions of the present invention to increase the solubility of poorly soluble substances and enhance the chemical stability of a drug. Suitable solvents/co-solvents, that may be employed, in the liquid compositions of the invention include, but are not limited to dichloromethane, acetonitrile, ethyl acetate, acetone, propylene carbonate, water, glycerin, coconut fatty acid diethanolamide, medium and/or long chain fatty acids or glycerides, monoglycerides, diglycerides, triglycerides, structured triglycerides, soyabean oil, peanut oil, corn oil, corn oil mono glycerides, corn oil di glycerides, corn oil triglycerides, polyethylene glycol, caprylocaproylmacroglycerides, caproyl 90, propylene glycol, propylene glycol esters, polyoxyethylenesorbitan fatty acid esters, polyoxyethylene castor oil derivatives, castor oil, cottonseed oil, olive oil, safflower oil, peppermint oil, coconut oil, palm seed oil, beeswax, oleic acid, methanol, ethanol, isopropyl alcohol, butanol, polyethylene alcohol, acetone, methyl isobutyl ketone, methyl ethyl ketone, N-methylpyrrolidone, dimethylacetamide, dimethylsulfoxide, dimethylisosorbide and the like or combinations thereof.

Surfactant is a general name for materials that possess surface activity; in solution they tend to orient at the surface of the liquid. There are several general classes of surfactants: anionic, cationic, amphoteric and non-ionic. Surfactants are amphiphilic molecules, i.e. part of the molecule is hydrophilic, and part is lipophilic. This combination of the two opposite affinities in the same molecule causes them to orient to the interface and thereby reduce the interfacial tension between the continuous and disperse phases, such as in emulsions and suspensions. Ionic surfactants work primarily through electrostatic forces, whereas non-ionic surfactants work primarily through steric forces. Non-limiting examples of surfactants are Sodium lauryl sulfate, Docusate sodium, Cocamidopropyl amino betaine, Polyoxyethylene sorbitan fatty acid esters (Polysorbate, Tween®), Polyoxyethylene 15 hydroxystearate (Macrogol 15 hydroxystearate, Solutol HS15®), Polyoxyethylene castor oil derivatives (Cremophor® EL, ELP, RH 40), Polyoxyethylene stearates (Myrj®), Sorbitan fatty acid esters (Span®), Polyoxyethylene alkyl ethers (Brij®), Polyoxyethylene nonylphenol ether (Nonoxynol®) and the like.

In some embodiments, the stabilizing agent is selected from sodium iodide, potassium iodide, polyvinylpyrrolidone, cross linked polyvinylpyrrolidone, methionine (preferably L-methionine or D-methionine or its pharmaceutically acceptable salt), sorbitol, and sorbitol solution. In some of the preferred embodiments, the stabilizing agent is selected from methionine (preferably L-methionine or D-methionine or its pharmaceutically acceptable salt), polyvinylpyrrolidone and a mixture thereof. The amount of methionine present in the Vancomycin compositions of the present invention ranges between about 10 mg and about 50 mg, preferably between about 15 mg and about 40 mg. By way of another example, the amount of methionine present in the compositions of the present invention ranges between about 1% by weight and about 5% by weight, preferably between about 1.5% by weight and about 4.0% by weight.

In some embodiments, a chelating agent is selected from edetate disodium (disodium EDTA), edetate disodium (disodium EDTA) anhydrous, edetate sodium, edetate calcium disodium, edetate calcium disodium anhydrous, edetic acid. In some of the preferred embodiments, a chelating agent is edetate disodium (disodium EDTA).

The specifically mentioned pharmaceutically acceptable excipients in the foregoing paragraphs are intended to be exemplary and not exhaustive of specific excipients that may be used in the practice of the disclosed invention. It is further understood that more than one of any particular type of excipient may be used in the compositions described herein. For example, the compositions may include more than one solvent/co-solvent/solubilizer, more than one antioxidant, more than one buffering agent and/or more than one pH adjusting/modifying agent, more than one preservative, more than one tonicity modifying agent, more than one stabilizing agent etc. and like. Also, a single excipient may provide multiple functions, as mentioned hereinabove.

The premixed formulations of the present invention can be packaged for use in a variety of containers. The formulations are preferably packaged in a pharmaceutically acceptable container, such as an intravenous bag or bottles. In order to avoid any degradation of the drug from light, packages can be used that reduce the amount of light which can reach the composition. For example, in some embodiments, the container may, optionally, further comprise a light barrier, such as an aluminum over pouch or a carton. In some of the further embodiments of the present invention, the premixed formulations are dispensed in intravenous bags, such as pre-mix bags and admix bags. Intravenous bags are well known in the art and commercially available.

In some of the alternative embodiments, pharmaceutically acceptable containers include intravenous bags, bottles, vials, and syringes. Preferred containers include intravenous bags and syringes, which are preferably polymer-based, and vials and intravenous bottles, which are preferably made of glass. It is also desirable to protect the formulations from light. Therefore, the container may, optionally, further comprise a light barrier. A preferred light barrier is an aluminum over pouch.

In some of the embodiments of the present invention, sterile formulations can be prepared using aseptic processing techniques. Sterility is maintained by using sterile materials and a controlled working environment. All containers and apparatus are sterilized, preferably by heat sterilization, prior to filling. Then, the container is filled under aseptic conditions, such as by passing the composition through a filter and filling the units. Therefore, the compositions can be sterile filled into a container to avoid the heat stress of terminal sterilization.

The order in which various components comprising the compositions is added to the solution is not critical, provided that the resulting compositions are stable and are suitable for continuous intravenous infusion. Accordingly, the compositions described herein can be made by preparing in a number of different ways. For example, in some embodiments, the compositions can be prepared by adding buffer, a tonicity agent and/or an anti-oxidant to water; adding Vancomycin to the buffered water solution; optionally adding a pH adjuster to achieve the desired pH; and then adding sufficient water to make up the final volume. If necessary, the pH can be readjusted to achieve the desired pH range. By way of another example, the compositions can be prepared by adding buffer and Vancomycin or a pharmaceutically acceptable salt thereof to water; adding a tonicity agent and/or an anti-oxidant, adjusting the pH to achieve the desired pH range; and then adding sufficient water to make up the final volume. By way of another example, an anti-oxidant can be added prior to the addition of Vancomycin or a pharmaceutically acceptable salt thereof, and a tonicity agent can be added after the addition of Vancomycin or a pharmaceutically acceptable salt thereof. By way of another example, a tonicity agent can be added prior to the addition of Vancomycin or a pharmaceutically acceptable salt thereof, and an antioxidant can be added after the addition of Vancomycin or a pharmaceutically acceptable salt thereof. By way of another example, the compositions can be prepared by adding buffer, tonicity agent and/or anti-oxidant to water; adjusting the pH to a first pH range suitable for dissolving Vancomycin or salt thereof; adding Vancomycin or a pharmaceutically acceptable salt thereof; adjusting the pH to achieve the desired final pH range; and then adding sufficient water to make up the final volume.

In some embodiments, pharmaceutical compositions comprising Vancomycin hydrochloride, sodium chloride, L-methionine and sodium hydroxide at pH 4.5-5.5 can be prepared by adding sodium chloride and L-methionine to water, adding Vancomycin hydrochloride, adjusting the pH if necessary to the range 4.5-5.5, and adding sufficient water to make up the final volume. If necessary, the pH can be readjusted to between about 4.5 to about 5.5.

In some embodiments, pharmaceutical compositions comprising Vancomycin hydrochloride, sodium chloride and sodium hydroxide at pH 4.5-5.5 can be prepared by adding sodium chloride to water, adding Vancomycin hydrochloride, adjusting the pH if necessary to the range 4.5-5.5, and adding sufficient water to make up the final volume. If necessary, the pH can be readjusted to between about 4.5 to about 5.5.

In some embodiments, pharmaceutical compositions comprising Vancomycin hydrochloride, sodium chloride, L-methionine and triethanolamine at pH 4.5-5.5 can be prepared by adding sodium chloride and L-methionine to water, adding Vancomycin hydrochloride, adjusting the pH if necessary to the range 4.5-5.5, and adding sufficient water to make up the final volume. If necessary, the pH can be readjusted to between about 4.5 to about 5.5.

Vancomycin hydrochloride may be added either directly to the solution containing sodium chloride and/or L-methionine or dissolved in water in separate vessel and then mixed with the solution containing sodium chloride and/or L-methionine. If the co-solvent is included in the formulation, the same can be used while solubilizing Vancomycin hydrochloride in separate vessel.

In some embodiments, the pre-mixed compositions of Vancomycin of the present invention exhibit improved or comparable bioavailability as compared to known Vancomycin compositions.

In some embodiments, the compositions are terminally sterilized using moist heat. Terminal sterilization can be used to destroy all viable microorganisms within the final, sealed container containing the pharmaceutical composition. An autoclave is typically used to accomplish terminal heat-sterilization of drug products in their final packaging.

Definitions:

The term “Vancomycin” as used herein also includes, the pharmaceutically acceptable salts, solvates, hydrates and anhydrous forms of Vancomycin. In particular, the liquid compositions of the present invention comprise Vancomycin or any pharmaceutically acceptable salt thereof. In one of the embodiments, the liquid compositions of the present invention comprise Vancomycin hydrochloride.

The term “pharmaceutically acceptable excipients” or “an excipient” as used herein refers to such pharmaceutically acceptable excipients which are known to those skilled in the art for the purposes of preparing parenteral or injectable pharmaceutical compositions. Such pharmaceutically acceptable excipients without limitation include stabilizing agents, chelating agents, anti-oxidants, surfactants and the like or combinations thereof. Such pharmaceutically acceptable excipients can be used in an amount which provides the compositions of the present invention desired property for which they are intended to use.

As used herein, the terms “stable” or “stability” encompass any characteristic of the premixed formulations which may be affected by storage conditions including, without limitation, potency, total impurities, degradation products, specific optical rotation, optical purity, water content, appearance, viscosity, sterility, and colour and clarity. The storage conditions which may affect stability include, for example, duration of storage, temperature, humidity, and/or light exposure.

The term “degradant”, “impurity”, “degradation impurity” and “related substance” as used herein represents the same meaning and can be used interchangeably.

In some of the embodiments of the present invention, “stable” or “storage stable”, or “stability” when used with reference to the premixed formulations of the present invention or when used “stable premixed formulations” or “stability of the premixed formulations” all these terms/phrases refer to formulations of the present invention which retain at least about 90%, or at least about 95%, or at least about 96%, or at least about 98%, of the labelled concentration of Vancomycin or salt thereof contained in the said formulation after storage under typical and/or accelerated conditions. In further embodiments, stable premixed formulations or stability of the premixed formulations refer to less than about 15% (area percent), or less than about 10% (area percent), or less than about 7% (area percent), or less than about 5% (area percent), or less than about 2% (area percent) of Vancomycin-related impurities are present after storage under typical and/or accelerated conditions.

In some of the embodiments, premixed formulations of the present invention contain no more than about 15% (area percent), or no more than about 10% (area percent), or no more than about 7% (area percent), or no more than about 5% (area percent), or no more than about 2% (area percent), or no more than about 1% (area percent), or no more than about 0.5% (area percent), or no more than about 0.2% (area percent), or no more than about 0.1% (area percent) any known or unknown single Vancomycin-related impurity or other impurity after storage under typical and/or accelerated conditions.

In some of the embodiments, premixed formulations of the present invention contain no more than about 15% (area percent), or no more than about 10% (area percent), or no more than about 7% (area percent), or no more than about 5% (area percent), or no more than about 2% (area percent), or no more than about 1% (area percent), or no more than about 0.5% (area percent), or no more than about 0.2% (area percent), or no more than about 0.1% (area percent) total Vancomycin-related impurities or other impurities after storage under typical and/or accelerated conditions.

Methods for determining the stability of the premixed formulations of the present invention with respect to a given parameter are well-known to those of skill in the art. For example, individual impurities and total impurities can be assessed by high-performance liquid chromatography (HPLC) or thin layer chromatography (TLC). Unless otherwise indicated to the contrary, a percentage amount of any individual impurities (known/unknown), or total impurities reported herein in the premixed formulations are determined by a peak area percent method using HPLC.

As used herein, “comprises”, “comprising”, “containing” and “having” and the like can have the meaning ascribed to them in patent law and can mean “includes”, “including” and the like, and are generally interpreted to be open ended terms. The terms “consisting of” or “consists of” are closed terms, and include only the components, structures, steps, or the like specifically listed in conjunction with such terms, as well as that which is in accordance with patent law. “Consisting essentially of” or “consists essentially of” have the meaning generally ascribed to them by patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open ended term, like “comprising” or “including” it is understood that direct support should be afforded also to “consisting essentially of” language as well as “consisting of” language as if stated explicitly and vice versa. In essence, use of one of these terms in the specification provides support for all of the others. The term “comprise/comprises/comprising” as used herein mean that other ingredients, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

“About” or “approximately” as used herein when referring to a measurable value such as an amount, a temporal duration, and the like, is meant to encompass variations of ±50%, ±40%, ±30%, ±20%, ±10%, more preferably ±5%, even more preferably ±1%, and still more preferably ±0.1% from the specified value, as such variations are appropriate to perform the disclosed methods. About also includes the exact amount. Hence “about 0.05 mg/mL” means “about 0.05 mg/mL” and also “0.05 mg/mL.” “About” or “approximately” is used to provide flexibility to a numerical value or range endpoint by providing that a given value may be “a little above” or “a little below” the value stated. “About” or “approximately” can mean, for example, within 3 or more than 3 standard deviations. “About” or “Approximately” can mean with an order of magnitude of a given value, for example, within 2-fold, 3-fold, 4-fold or 5-fold of a value. However, it is to be understood that even when a numerical value is accompanied by the term “about” in this specification, that express support shall be provided at least for the exact numerical value as well as though the term “about” were not present.

The use of the terms “a” and “an” and “the” and similar referents in the context of describing the invention are to be construed to cover both the singular and the plural, unless otherwise indicated herein or clearly contradicted by context.

The term “compatible” as used herein refers to those added excipients or ingredients or additives those are not substantially antagonistic to the other excipients or ingredients or additives or pharmaceutically active ingredients.

As used herein, “treatment” refers to ameliorating or reducing symptoms associated with a disease or condition. Treatment means any manner in which the symptoms of a condition, disorder or disease are ameliorated or otherwise beneficially altered. Hence treatment encompasses prophylaxis, therapy and/or cure. Treatment also encompasses any pharmaceutical use of the compositions herein.

As used herein, “treating” a subject having a disease or condition means that a composition or other product provided or described herein is administered to the subject to thereby effect treatment thereof.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, “prevention” or “prophylaxis” refers to methods in which the risk of developing disease or condition is reduced. Prophylaxis includes reduction in the risk of developing a disease or condition and/or a prevention of worsening of symptoms or progression of a disease, or reduction in the risk of worsening of symptoms or progression of a disease.

As used herein an “effective amount” of a compound or composition for treating a particular disease is an amount that is sufficient to ameliorate, or in some manner reduce symptoms to achieve the desired physiological effect. Such amount can be administered as a single dosage or can be administered according to a regimen, whereby it is effective. The effective amount is readily determined by one of skill in the art following routine procedures.

As used herein, “disease” or “disorder” or “condition” refers to a pathological condition in an organism resulting from cause or condition including, but not limited to, infections, acquired conditions, genetic conditions, and characterized by identifiable symptoms.

As used herein, “patient” or “subject” to be treated includes humans and or non-human animals, including mammals. Mammals include primates, such as humans, chimpanzees, gorillas and monkeys; domesticated animals, such as dogs, horses, cats, pigs, goats, cows; and rodents such as mice, rats, hamsters and gerbils.

As used herein, “optional” or “optionally” means that the subsequently described event or circumstance does or does not occur, and that the description includes instances where said event or circumstance occurs and instances where it does not. For example, an optionally substituted group means that the group is un-substituted or is substituted.

All percentages mentioned herein, unless otherwise indicated, are on a w/v basis, i.e. percentage ingredient (active/inactive) present by weight in the total volume of the composition.

“Effective dosage amount” as used herein with respect to, for example Vancomycin premixed formulations shall mean that dosage that provides the specific pharmacological response for which Vancomycin or salt thereof administered in a significant number of subjects in need of such treatment. It is emphasized that “effective dosage amount”, administered to a particular subject in a particular instance will not always be effective in treating the diseases described herein, even though such dosage is deemed a “effective dosage amount” by those skilled in the art.

As used herein, the term “pre-mixed” refers to a pharmaceutical composition that does not require reconstitution or dilution before administration to a patient. In contrast to vial formulations comprising Vancomycin hydrochloride that must be diluted prior to use in a diluent and container selected by hospital personnel. Alternatively, the term “pre-mixed” may also mean a pharmaceutical composition wherein the liquid solution and the active pharmaceutical ingredient are separated from the point of manufacture and in storage, such as when the solution is stored in an intravenous bag and the active pharmaceutical ingredient is lyophilized and stored in a vial that is connected to the bag, but not in fluid contact with the solution until just before administration to a patient. Preferably, the pharmaceutical compositions are aqueous solutions that are administered by injection. Alternatively, the pharmaceutical compositions may be lyophilized and then reconstituted in isotonic saline, for example, before intravenous administration.

As used herein, the term “ready-to-use” refers to a pharmaceutical composition which can be administered directly to the patients in need thereof without prior reconstitution and/or dilution.

The present invention is further exemplified by following non-limiting examples.

### EXAMPLES

The compositions of the present invention are explained in more detail with reference to the following examples. These examples are provided by way of illustration only and should not be construed as to limit the scope or spirit of the appended claims in any manner.

| Name of Ingredient | | | | | | | | | | | |
|---|---|---|---|---|---|---|---|---|---|---|---|
| (Role of ingredient) | Example-1 | | Example-2 | | Example-3 | | Example-4 | | Example-5 | Example-6 | |
| Vancomycin^{#} | 5 | mg/mL | 5 | mg/mL | 5 | mg/mL | 5 | mg/mL | 5 mg/mL | 5 | mg/mL |
| (Active) | | | | | | | | | | | |
| Sodium chloride | 8 | mg/mL | 8 | mg/mL | 1 | mg/mL | 1 | mg/mL | 1 mg/mL | 8 | mg/mL |
| (Tonicity modifying agent) | | | | | | | | | | | |

| | | | | | | |
|---|---|---|---|---|---|---|
| Dextrose | — | — | — | — | — | — |
| (Tonicity modifying agent) | | | | | | |

| | | | | | | | | | | | |
|---|---|---|---|---|---|---|---|---|---|---|---|
| L-methioninc | 20 | mg/mL | 40 | mg/mL | 40 | mg/mL | 40 | mg/mL | — | 20 | mg/mL |
| (Anti-oxidant/stabilizer) | | | | | | | | | | | |

| | | | | | | |
|---|---|---|---|---|---|---|
| Sodium hydroxide | Q.S.* | Q.S.* | Q.S.* | Q.S.* | Q.S.* | — |
| (pH adjusting agent) | | | | | | |
| Triethanolamine | — | — | — | — | — | Q.S.* |
| (Buffer/pH adjusting agent) | | | | | | |
| Water for injection | Q.S.^{@} | Q.S.^{@} | Q.S.^{@} | Q.S.^{@} | Q.S.^{@} | Q.S.^{@} |
| (Vehicle) | | | | | | |

^{#}Equivalent amount of Vancomycin hydrochloride is used after potency correction
Q.S.*= Quantity sufficient to adjust/maintain pH between about 4.0 and about 6.0
Q.S.^{@}= Quantity sufficient to 1 mL
Notes:
1. The vancomycin formulation prepared in Example-1 by following two part method at
25° C. was filed into 100 mL SGD vial and kept al 2-8° C.
2. The vancomycin formulation prepared in Example-3 by following two part method at
25° C. was filled into 100 mL SGD vial and kept at 2-8° C.
3. The vancomycin formulation prepared in Example-4 by following two part method at
25° C. was filled into 250 mL polar polymer free infusion bags and was kept at 2-8° C.
4. The vancomycin formulation prepared in Example-5 by following two part method at
2-8° C. was filled into 100 mL SGD vial and kept at 2-8° C.

| Name of Ingredient | | | | | | |
|---|---|---|---|---|---|---|
| (Role of ingredient) | Example-7 | Example-8 | Example-9 | Example-10 | Example-11 | Example-12 |
| Vancomycin^{#} (Active) | 5 mg/mL | 5 mg/mL | 5 mg/mL | 5 mg/mL | 5 mg/mL | 5 mg/mL |
| Mannitol | — | — | 20 mg/mL | — | 20 mg/mL | — |
| (Tonicity modifying agent) | | | | | | |
| L-methionine | 20 mg/mL | — | 20 mg/mL | — | — | — |
| (Stabilizer) | | | | | | |
| Polyvinyl pyrrolidone | — | — | — | 20 mg/mL | 20 mg/mL | — |
| (Stabilizer) | | | | | | |
| Polyethylene glycol-400 | — | 20 mg/mL | 20 mg/mL | — | 20 mg/mL | — |
| (co-solvent/solubilizer) | | | | | | |
| Poloxamer | — | — | — | — | — | 6 mg/mL |
| (co-solvent/solubilizer) | | | | | | |
| Acetate buffer^{$} | Q.S.* | Q.S.* | Q.S.* | Q.S.* | Q.S.* | — |
| Sodium lactate | — | — | — | — | — | Q.S.* |
| (pH adjusting agent) | | | | | | |
| Edetate disodium | — | — | — | — | — | — |
| (Chelating agent) | | | | | | |
| Sodium hydroxide | — | — | — | — | — | — |
| (pH adjusting agent) | | | | | | |
| Acetic acid | — | — | — | — | — | — |
| (pH adjusting agent) | | | | | | |
| Sodium acetate trihydrate | — | — | — | — | — | — |
| (Buffering agent) | | | | | | |

^{#}Equivalent amount of Vancomycin hydrochloride is used after potency correction
Q.S.*= Quantity sufficient to adjust/maintain pH between about 4.0 and about 6.0
Q.S.^{@}= Quantity sufficient to 1 mL
^{$}Acetate buffer comprises glacial acetic acid and sodium acetate trihydrate

| Name of Ingredient | | | | | | | | | |
|---|---|---|---|---|---|---|---|---|---|
| (Role of ingredient) | Example-13 | | Example-14 | Example-15 | | Example-16 | | Example-17 | |
| Vancomycin^{#} (Active) | 5 | mg/mL | 5 mg/mL | 5 | mg/mL | 5 | mg/mL | 5 | mg/mL |

| | | | | | | | | |
|---|---|---|---|---|---|---|---|---|
| Mannitol | — | — | 20 | mg/mL | 20 | mg/mL | 20 | mg/mL |
| (Tonicity modifying agent) | | | | | | | | |

| | | | | | | | | | |
|---|---|---|---|---|---|---|---|---|---|
| L-methionine | 20 | mg/mL | — | 15 | mg/mL | 15 | mg/mL | 15 | mg/mL |
| (Stabilizer) | | | | | | | | | |

| Polyvinyl pyrrolidone | — | — | — | | — | | 2 | mg/mL |
|---|---|---|---|---|---|---|---|---|
| (Stabilizer) | | | | | | | | |
| Polyethylene glycol-400 | — | — | 15 | mg/mL | 15 | mg/mL | 15 | mg/mL |
| (co-solvent/solubilizer) | | | | | | | | |
| Poloxamer | — | — | — | | — | | — | |
| (co-solvent/solubilizer) | | | | | | | | |
| Acetate buffer^{$} | — | Q.S.* | — | | — | | — | |
| Sodium lactate | — | — | — | | — | | — | |
| (pH adjusting agent) | | | | | | | | |
| Edetate disodium | — | 1 mg/mL | — | | — | | — | |
| (Chelating agent) | | | | | | | | |
| Sodium hydroxide | Q.S.* | — | — | | — | | — | |
| (pH adjusting agent) | | | | | | | | |
| Acetic acid⁺ | — | — | Q.S.* | | Quantity sufficient | | Q.S.* | |
| (pH adjusting agent) | | | | | to adjust/maintain | | | |
| | | | | | pH between about 3.0 | | | |
| | | | | | and about 6.0 | | | |
| Sodium acetate trihydrate | — | — | 1 | mg/mL | 1 | mg/mL | 1 | mg/mL |
| (Buffering agent) | | | | | | | | |

^{#}Equivalent amount of Vancomycin hydrochloride is used after potency correction
Q.S.*= Quantity sufficient to adjust/maintain pH between about 4.0 and about 6.0
Q.S.^{@}= Quantity sufficient to 1 mL
^{$}Acetate buffer comprises glacial acetic acid and sodium acetate trihydrate
⁺used as either 10% v/v solution or 20% v/v solution

| Name of Ingredient | | | | | | | | | | | | | | |
|---|---|---|---|---|---|---|---|---|---|---|---|---|---|---|
| (Role of ingredient) | Ex-18 | | Ex-19 | | Ex-20 | | Ex-21 | | Ex-22 | | Ex-23 | | Ex-24 | |
| Vancomycin* (Active) | 50 | mg/mL | 50 | mg/mL | 50 | mg/mL | 50 | mg/mL | 50 | mg/mL | 50 | mg/mL | 50 | mg/mL |
| Sodium chloride | 8 | mg/mL | 8 | mg/mL | 1 | mg/mL | 1 | mg/mL | 8 | mg/mL | 1-8 | mg/mL | 1-8 | mg/mL |
| (Tonicity modifying agent) | | | | | | | | | | | | | | |

| Dextrose | — | | — | | — | | — | | — | | — | — |
|---|---|---|---|---|---|---|---|---|---|---|---|---|
| (Tonicity modifying agent) | | | | | | | | | | | | |
| L-methionine | 20 | mg/mL | 40 | mg/mL | 40 | mg/mL | 40 | mg/mL | 20 | mg/mL | — | — |
| (Anti-oxidant/stabilizer) | | | | | | | | | | | | |

| | | | | | | | |
|---|---|---|---|---|---|---|---|
| Sodium hydroxide | Q.S.* | Q.S.* | QS.* | Q.S.* | — | Q.S.* | — |
| (pH adjusting agent) | | | | | | | |
| Triethanolamine | — | — | — | — | Q.S.* | — | Q.S.* |
| (Buffer/pH adjusting agent) | | | | | | | |
| Water for injection | Q.S.^{@} | Q.S.^{@} | Q.S.^{@} | Q.S.^{@} | Q.S.^{@} | Q.S.^{@} | Q.S.^{@} |
| (Vehicle) | | | | | | | |

^{#}Equivalent amount of Vancomycin hydrochloride is used
Q.S.*= Quantity sufficient to adjust/maintain pH between about 4.0 and about 6.0
Q.S.@= Quantity sufficient to 1 mL

Stability Study Data of Pre-Mixed, Ready-to-Use Vancomycin Formulations:

Storage condition: 2-8° C.

| | Example-1 | | | | | Example-3 | | |
|---|---|---|---|---|---|---|---|---|
| | Initial | 1 M | 3 M | 6 M | 12 M | Initial | 3 M | 6 M |
| | Description | | | | | | | |
| Test | Clear colorless solution | | | | | Clear colorless solution | | |
| Vancomycin-B (%) | 94.85 | 93.65 | 90.81 | 87.84 | 82.22 | 94.99 | 91.44 | 87.92 |
| Resolution compound 1 | 1.16 | 1.62 | 1.88 | 2.21 | 2.17 | 1.11 | 1.96 | 2.35 |
| (%) (CDP-1-m) | | | | | | | | |
| Resolution compound 2 | 1.50 | 1.72 | 2.85 | 4.15 | 7.71 | 1.45 | 2.52 | 3.83 |
| (%) (CDP-1-M) | | | | | | | | |

Storage condition: 2-8° C.

| | Example-4 | | | | Example-5 | | | |
|---|---|---|---|---|---|---|---|---|
| | Initial | 1 M | 3 M | 6 M | Initial | 1 M | 3 M | 6 M |
| | Description | | | | | | | |
| Test | Clear colorless solution | | | | Clear colorless solution | | | |
| Vancomycin-B (%) | 95.63 | 92.60 | 91.18 | 88.20 | 95.31 | 93.50 | 92.02 | 89.31 |
| Resolution compound 1 | 0.46 | 1.63 | 2.13 | 2.49 | 0.38 | 1.02 | 1.39 | 1.66 |
| (%) (CDP-1-m) | | | | | | | | |
| Resolution compound 2 | 1.32 | 1.69 | 2.62 | 4.18 | 1.29 | 1.49 | 2.60 | 3.95 |
| (%) (CDP-1-M) | | | | | | | | |

Storage condition: 2-8° C.

| | Example-15 | | Example-16 | |
|---|---|---|---|---|
| Test | Initial | 1M | Initial | 1M |
| Description | Clear colorless solution | | Clear colorless solution | |
| Vancomycin-B (%) | 91.40 | 91.26 | 91.82 | 90.50 |
| Resolution | 1.50 | 1.45 | 1.26 | 1.78 |
| compound 1 | | | | |
| (%) (CDP-1-m) | | | | |
| Resolution | 1.93 | 2.04 | 1.81 | 2.21 |
| compound 2 | | | | |
| (%) (CDP-1-M) | | | | |

Methods of Preparation:

The pharmaceutical compositions comprising Vancomycin hydrochloride, sodium chloride, L-methionine and sodium hydroxide at pH 4.5-5.5 can be prepared by adding sodium chloride and L-methionine to water, adding Vancomycin hydrochloride, adjusting the pH if necessary to the range 4.5-5.5, and adding sufficient water to make up the final volume. If necessary, the pH can be readjusted to between about 4.5 to about 5.5.

The pharmaceutical compositions comprising Vancomycin hydrochloride, sodium chloride and sodium hydroxide at pH 4.5-5.5 can be prepared by adding sodium chloride to water, adding Vancomycin hydrochloride, adjusting the pH if necessary to the range 4.5-5.5, and adding sufficient water to make up the final volume. If necessary, the pH can be readjusted to between about 4.5 to about 5.5.

The pharmaceutical compositions comprising Vancomycin hydrochloride, sodium chloride, L-methionine and triethanolamine at pH 4.5-5.5 can be prepared by adding sodium chloride and L-methionine to water, adding Vancomycin hydrochloride, adjusting the pH if necessary to the range 4.5-5.5, and adding sufficient water to make up the final volume. If necessary, the pH can be readjusted to between about 4.5 to about 5.5.

Vancomycin hydrochloride may be added either directly to the solution containing sodium chloride and/or L-methionine or dissolved in water in separate vessel and then mixed with the solution containing sodium chloride and/or L-methionine.

The pharmaceutical composition comprising Vancomycin hydrochloride, L-methionine and acetate buffer at pH 4.5-5.5 as described in Example—7 can be prepared by adding L-methionine to water, adding Vancomycin hydrochloride, adjusting the pH if necessary to the range 4.5-5.5, and adding sufficient water to make up the final volume. If necessary, the pH can be readjusted to between about 4.5 and about 5.5. Similarly, other examples 8-14 can be practiced by using suitable excipients mentioned in table above.

The pharmaceutical compositions described in Examples 15-17 can be prepared by step by step adding required quantities of L-methionine, polyethylene glycol, mannitol, polyvinyl pyrrolidone (where required), Vancomycin hydrochloride and sodium acetate trihydrate, adjusting the pH if necessary to the range 3.5-5.5, and adding sufficient water to make up the final volume. If necessary, the pH can be readjusted to between about 3.5 and about 5.5.

The pharmaceutical compositions of the present invention as described herein are suitable for making or using in the industry.

It should be understood that various changes and modifications to the various aspects/embodiments described herein will be apparent to those skilled in the art. Such changes and modifications can be made without departing from the spirit and scope of the subject matter of the present invention and without diminishing its intended advantages. It is therefore intended that such changes and modifications be covered within the scope of the present invention and claims.

Structures, substances, excipients, methods etc. that perform substantially the same function in substantially the same way to achieve substantially the same results are included within the scope of the claims.

What is claimed is:

1. A vancomycin composition comprising:
(i) about 0.1 mg/mL to about 50 mg/mL vancomycin or its pharmaceutically acceptable salt; and
(ii) at least one of the following:
(a) a stabilizer selected from methionine, polyvinylpyrrolidone and a mixture thereof;
(b) a solubilizer/co-solvent selected from polyethylene glycol, poloxamer and a mixture thereof;
(c) a tonicity modifying agent;
(d) a chelating agent selected from edetate disodium (disodium EDTA), edetate disodium (disodium EDTA) anhydrous, edetate sodium, edetate calcium disodium, edetate calcium disodium anhydrous, edetic acid and a mixture thereof;
(e) a buffering agent and/or a pH adjusting agent; and
(f) any combination of (a), (b), (c), (d) and (e).

2. The vancomycin composition as claimed in claim 1, wherein
a tonicity modifying agent is selected from sodium chloride, dextrose, sucrose, xylitol, fructose, glycerol, sorbitol, mannitol, potassium chloride, mannose, calcium chloride, magnesium chloride and a mixture thereof;
a buffering agent is selected from arginine, alanine, histidine, glycine, lysine, citrate, glutamate, bicarbonate, tartrate, benzoate, lactate, gluconate, triethanolamine, trolamine, acetate, meglumine, borate, phosphate, ammonium phosphate, diethanolamine, potassium acetate, potassium citrate, potassium metaphosphate, potassium phosphate, sodium acetate, sodium citrate, sodium glycolate, sodium lactate, sodium phosphate and a mixture thereof; and
a pH adjusting agent is selected from sodium hydroxide, potassium hydroxide, calcium hydroxide, sodium carbonate, sodium bicarbonate, potassium carbonate, potassium bicarbonate, calcium carbonate, magnesium carbonate, lithium carbonate, cesium carbonate and a mixture thereof.

3. The vancomycin composition as claimed in claim 1, wherein said composition has pH between about 3.0 and about 6.0.

4. The vancomycin composition as claimed in claim 1, wherein said composition comprises:
about 0.1 mg/mL to about 50 mg/mL vancomycin or its pharmaceutically acceptable salt; and
a combination of (ii)(a) and (ii)(e).

5. The vancomycin composition as claimed in claim 1, wherein said composition comprises:
about 0.1 mg/mL to about 50 mg/mL vancomycin or its pharmaceutically acceptable salt; and
a combination of (ii)(b) and (ii)(e).

6. The vancomycin composition as claimed in claim 1, wherein said composition comprises:
about 0.1 mg/mL to about 50 mg/mL vancomycin or its pharmaceutically acceptable salt; and
a combination of (ii)(a), (ii)(c) and (ii)(e).

7. The vancomycin composition as claimed in claim 1, wherein said composition comprises:
about 0.1 mg/mL to about 50 mg/mL vancomycin or its pharmaceutically acceptable salt; and
a combination of (ii)(a), (ii)(b), (ii)(c) and (ii)(e).

8. The vancomycin composition as claimed in claim 1, wherein said composition comprises:
about 0.1 mg/mL to about 50 mg/mL vancomycin or its pharmaceutically acceptable salt; and
a combination of (ii)(d) and (ii)(e).

9. The vancomycin composition as claimed in claim 1, wherein said composition comprises:
about 0.1 mg/mL to about 50 mg/mL vancomycin or its pharmaceutically acceptable salt; and
a combination of (ii)(a), (ii)(b), (ii)(c), (ii)(d) and (ii)(e).

10. A vancomycin composition comprising about 0.1 mg/mL to about 50 mg/mL vancomycin or its pharmaceutically acceptable salt and methionine.

11. The vancomycin composition as claimed in claim 10, wherein methionine is selected from L-methionine, D-methionine or its pharmaceutically acceptable salts.

12. The vancomycin composition as claimed in claim 11, wherein methionine is L-methionine or its pharmaceutically acceptable salt.

13. The vancomycin composition as claimed in claim 10, wherein said composition has pH between about 3.0 and about 6.0.

14. The vancomycin composition as claimed in claim 10, wherein said composition further comprises a pharmaceutically acceptable excipient selected from a tonicity modifying agent, a chelating agent, a solubilizer/co-solvent, a buffering agent, a pH adjusting agent, a stabilizing agent and combinations thereof.

15. The vancomycin composition as claimed in claim 14, wherein
a tonicity modifying agent is selected from sodium chloride, dextrose, sucrose, xylitol, fructose, glycerol, sorbitol, mannitol, potassium chloride, mannose, calcium chloride, magnesium chloride and a mixture thereof;
a chelating agent is selected from edetate disodium (disodium EDTA), edetate disodium (disodium EDTA) anhydrous, edetate sodium, edetate calcium disodium, edetate calcium disodium anhydrous, edetic acid and a mixture thereof;
a solubilizer/co-solvent is selected from polyethylene glycol, propylene glycol, ethanol, poloxamer, sorbitol, mannitol, xylitol and a mixture thereof;
a buffering agent is selected from arginine, alanine, histidine, glycine, lysine, citrate, glutamate, bicarbonate, tartrate, benzoate, lactate, gluconate, triethanolamine, trolamine, acetate, meglumine, borate, phosphate, ammonium phosphate, diethanolamine, potassium acetate, potassium citrate, potassium metaphosphate, potassium phosphate, sodium acetate, sodium citrate, sodium glycolate, sodium lactate, sodium phosphate and a mixture thereof;
a pH adjusting agent is selected from sodium hydroxide, potassium hydroxide, calcium hydroxide, sodium carbonate, sodium bicarbonate, potassium carbonate, potassium bicarbonate, calcium carbonate, magnesium carbonate, lithium carbonate, cesium carbonate and a mixture thereof; and
a stabilizing agent is selected from sodium iodide, potassium iodide, polyvinylpyrrolidone, cross linked polyvinylpyrrolidone, sorbitol, sorbitol solution and a mixture thereof.

16. A vancomycin composition comprising about 0.1 mg/mL to about 50 mg/mL vancomycin or its pharmaceutically acceptable salt and a pharmaceutically acceptable excipient selected from a tonicity modifying agent, a solubilizer/co-solvent, a buffering agent and a pH adjusting agent.

17. The vancomycin composition as claimed in claim 16, wherein said composition does not include a stabilizing agent.

18. The vancomycin composition as claimed in claim 16, wherein
a tonicity modifying agent is selected from sodium chloride, dextrose, sucrose, xylitol, fructose, glycerol, sorbitol, mannitol, potassium chloride, mannose, calcium chloride, magnesium chloride and a mixture thereof;
a solubilizer/co-solvent is selected from polyethylene glycol, propylene glycol, ethanol, poloxamer, sorbitol, mannitol, xylitol and a mixture thereof;
a buffering agent is selected from arginine, alanine, histidine, glycine, lysine, citrate, glutamate, bicarbonate, tartrate, benzoate, lactate, gluconate, triethanolamine, trolamine, acetate, meglumine, borate, phosphate, ammonium phosphate, diethanolamine, potassium acetate, potassium citrate, potassium metaphosphate, potassium phosphate, sodium acetate, sodium citrate, sodium glycolate, sodium lactate, sodium phosphate and a mixture thereof; and
a pH adjusting agent is selected from sodium hydroxide, potassium hydroxide, calcium hydroxide, sodium carbonate, sodium bicarbonate, potassium carbonate, potassium bicarbonate, calcium carbonate, magnesium carbonate, lithium carbonate, cesium carbonate and a mixture thereof.
